(19) United States
(12) Patent Application Publication
(10) Pub. No.: US 2004/0146868 A1
(43) Pub. Date: Jul. 29, 2004

(54) Methods and nucleic acids for the analysis of CpG dinucleotide methylation status associated with the development of peripheral zone prostate cancer

(75) Inventors: Susan Cottrell, Seattle, WA (US); Andrew Sledziewski, Shoreline, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Epigenomics AG, Berlin (DE)

(21) Appl. No.: 10/350,763

(22) Filed: Jan. 24, 2003

(51) Int. Cl. C12Q 1/68

(52) U.S. Cl. 435/6

(57) ABSTRACT

The present invention provides for molecular GSTP1 markers that have novel utility for the analysis of methylation patterns within the promoter region and exons 1 and 2 of the GSTP1 gene, and are further useful in methods to effectively distinguish among benign hyperplasia of the prostate and different grades of prostate cancer. Additionally, the subject molecular GSTP1 markers have novel utility for the precise localization of the zone of origin to provide sensitive, accurate and non-invasive methods for the diagnosis and/or prognosis of prostate cell proliferative disorders. The present invention has novel utility for the detection and differentiation of a cell proliferative disorder of the peripheral zone of the prostate.

### FIELD OF THE INVENTION

The present invention relates to human DNA sequences that exhibit altered methylation patterns (hypermethylation or hypomethylation) in cancer patients. Particular embodiments of the invention provide highly accurate methods for detection and differentiation of peripheral zone prostate carcinomas.

### BACKGROUND

Correlation of aberrant DNA methylation with cancer. Aberrant DNA methylation within CpG ‘islands’ is characterized by hyper- or hypomethylation of CpG dinucleotide sequences leading to abrogation or overexpression of a broad spectrum of genes, and is among the earliest and most common alterations found in, and correlated with human malignancies. Additionally, abnormal methylation has been shown to occur in CpG-rich regulatory elements in intronic and coding parts of genes for certain tumors. In colon cancer, aberrant DNA methylation constitutes one of the most prominent alterations and inactivates many tumor suppressor genes including, inter alia, p14ARF, p161NK4a, THBS1, MINT2, and MINT31 and DNA mismatch repair genes such as hMLH1.

Aside from the specific hypermethylation of tumor suppressor genes, an overall hypomethylation of DNA can be observed in tumor cells. This decrease in global methylation can be detected early, far before the development of frank tumor formation. A correlation between hypomethylation and increased gene expression has been determined for many oncogenes.

Prostate cancer. The prostate is a male sex accessory gland, comprising about 30 to 50 branched glands. It is surrounded by a fibroelastic capsule that separates the gland into discrete lobes. The central zone of the organ is composed of pseudo stratified epithelium, the peripheral zone comprises the bulk of the organ and the two tissue types are separated by a transitional zone.

Benign prostate hypertrophy is present in about 50% of men aged 50 or above, and in 95% of men aged 75 or above. Prostate cancer is a significant health care problem in Western countries with an incidence of 180 per 100,000 in the United States in 1999 (Cancer J. Clin., 49:8, 1999). Neoplasms arising in the transitional zone are considered to have less malignant potential than those arising in the peripheral zone. Analysis of the two tissue types indicates that compared to peripheral zone cancers (PZ cancers), tumors originating in the transitional zone of the prostate (TZ carcinomas) exhibit lower Gleason scores and lower expression of markers related to tumour growth, which might contribute to a less malignant clinical behavior (see, e.g., Henke et al., Eur. Urol., 41:40-6, 2002; “Tumour grade, proliferation, apoptosis, microvessel density, p53, and bcl-2 in prostate cancers: differences between tumours located in the transition zone and in the peripheral zone”).

Diagnosis and prognosis of prostate cancer; deficiencies of prior art approaches. Different screening strategies have been employed with at least some degree of success to improve early detection of prostate cancer, including determination of levels of prostate specific antigen (“PSA”) and digital rectal examination. If a prostate carcinoma is suspected in a patient, diagnosis of cancer is confirmed or excluded by the histological and cytological analysis of biopsy samples for features associated with malignant transformation. The zone of origin of a prostatic cell proliferative disorder is currently determined by the ‘PSA density.’ PSA density is determined by dividing the weight of the prostate (as estimated by transrectal ultrasound) by the prostate specific antigen levels of the patient. Levels of over 15% percent are considered as indicative of prostate cancer and grounds for a biopsy. The biopsy, in turn, is used for histological and cytological analysis to determine the zone of origin.

However, using routine histological examination, it is often difficult to distinguish benign hyperplasia of the prostate from early stages of prostate carcinoma, even if an adequate biopsy is obtained (McNeal J. E. et al., Hum. Pathol. 2001, 32:441-6). Furthermore, small or otherwise insufficient biopsy samples often impede the analysis.

Molecular markers would offer the advantage that they could be used to efficiently analyze even very small tissue samples, and samples whose tissue architecture has not been maintained. Within the last decade, numerous genes have been studied with respect to differential expression among benign hyperplasia of the prostate and different grades of prostate cancer.

However, no single marker has as yet been shown to be sufficient for the distinction between the two lesions or for determination of the zone of origin.

Alternatively, high-dimensional mRNA based approaches may, in particular instances, provide a means to distinguish between different tumor types and benign and malignant lesions. However, application of such approaches as a routine diagnostic tool in a clinical environment is impeded and substantially limited by the extreme instability of mRNA, the rapidly occurring expression changes following certain triggers (e.g., sample collection), and, most importantly, by the large amount of mRNA needed for analysis which often cannot be obtained from a routine biopsy (see, e.g., Lipshutz, R. J. et al., Nature Genetics 21:20-24, 1999; Bowtell, D. D. L. Nature Genetics Suppl. 21:25-32, 1999). Here again, however, there is no provision for determination of the zone of origin.

The GSTP1 gene. The core promoter region of the Gluthione S-Transferase P gene (GSTP1; accession no. NM_{—}000852) has been shown to be hypermethylated in prostate tumor tissue. The glutathione S-transferase pi enzyme is involved in the detoxification of electrophilic carcinogens, and impaired or decreased levels of enzymatic activity (GSTPi impairment) have been associated with the development of neoplasms, particularly in the prostate. Mechanisms of GSTPi impairment include mutation (the GSTP*B allele has been associated with a higher risk of cancer) and methylation.

Prior art GSTP1 studies. Expression levels of the GSTP1 gene have been measured comparatively in high grade prostatic intraepithelial neoplasia of the transitional and peripheral zones by means of immunohistological staining (Bartels et. al., Mol Pathol., 53:122-8, 2000; “Expression of pi-class glutathione S-transferase: two populations of high grade prostatic intraepithelial neoplasia with different relations to carcinoma”). The two types of tissues had distinct expression patterns. In the transitional zone, neoplasia staining was similar to that of normal tissue, whereas in the peripheral zone, neoplasia staining was characterised by a lack of GST-Pi expression in the secretory cells and abundant expression in the scattered basal cells.

Lee et al., in U.S. Pat. No. 5,552,277, disclosed that the expression of the gluthione-S-transferase (GST) Pi gene was downregulated in a significant proportion of prostate carcinomas. Moreover, by means of restriction enzyme analysis they were able to show that the promoter region of the of the GSTPi gene was upmethylated (hypermethylated) in prostate carcinomas as opposed to normal prostate and leukocyte tissue. However, due to the limited and imprecise nature of the analysis technique used (HpaIII digestion, followed by Southern blotting) the exact number and position of the methylated CG dinucleotides were not characterized.

Douglas et al. (WO9955905) used a method comprising bisulfite treatment, followed by methylation specific PCR to show that prostate carcinoma-specific GSTPi hypermethylation was localized to the core promoter regions, and localized a number of CpG positions that had not been characterised by Lee et al.

Herman and Baylin (U.S. Pat. No. 6,017,704) describe the use of methylation specific primers for methylation analysis, and describe a particular primer pair suitable for the analysis of the corresponding methylated GSTPi promoter sequence.

However, with respect to the use of GSTPi markers, the prior art is limited with respect to the number of GSTPi promoter CpG sequences that have been characterized for differential methylation status. Moreover, there are no disclosures, suggestions or teachings in the prior art of how such markers could be used to distinguish among benign hyperplasia of the prostate and different grades of prostate cancer. Furthermore, there are no disclosures, suggestions or teachings in the prior art of how such markers might be used to determine the zone of origin to improve diagnostic and prognostic analyses, and/or to obviate the need for histological analyses of biopsies.

Pronounced need in the art. Therefore, in view of the incidence of prostate hyperplasia (50% of men aged 50 or above, and 95% of men aged 75 or above) and prostate cancer (180 per 100,000), there is a substantial need in the art for the development of molecular markers that could be used to effectively distinguish among benign hyperplasia of the prostate and different grades of prostate cancer. Additionally, there is a pronounced need in the art for the development of molecular markers that could be used for the precise localization of the zone of origin to provide sensitive, accurate and non-invasive methods (as opposed to, e.g., biopsy and transrectal ultrasound) for the diagnosis, prognosis and treatment of prostate cell proliferative disorders.

### SUMMARY OF THE INVENTION

The present invention provides novel uses for the analysis of differential methylation patterns within the GSTP1 gene promoter region (SEQ ID NO:1) that includes exons one and two. Such novel uses include diagnostic and prognostic assays for cancer, based on measurement of differential methylation of GSTP1-specific CpG dinucleotide sequences between test and control samples.

Particular embodiments enable detection and differentiation between a cell proliferative disorders of the transitional zone and the peripheral zone of the prostate. Identification of the zone of origin of the prostate cell proliferative disorders is directly linked with disease prognosis, and the method according to the invention thereby enables the physician and patient to make better and more informed treatment decisions.

Preferably, the source of the test sample is selected from the group consisting of cell lines, histological slides, biopsies, paraffin-embedded tissue, bodily fluids, ejaculate, urine, blood, and combinations thereof. Preferably, the source is biopsies, bodily fluids, ejaculate, urine, or blood.

Specifically, the present invention provides a method for detecting a cell proliferative disorder of a prostate peripheral zone, or for distinguishing between a transitional and a peripheral zone of origin of a prostate cell proliferative disorder, comprising: obtaining a biological sample having genomic nucleic acid; contacting the nucleic acid, or a fragment thereof, with one reagent or a plurality of reagents sufficient for distinguishing between methylated and non methylated CpG dinucleotide sequences within a target sequence of the subject nucleic acid, wherein the target sequence comprises, or hybridizes under stringent conditions to, at least 18 contiguous nucleotides of SEQ ID NO:1, said contiguous nucleotides comprising at least one CpG dinucleotide sequence; and determining, based at least in part on said distinguishing, the methylation state of at least one target CpG dinucleotide sequence, or an average, or a value reflecting an average methylation state of a plurality of target CpG dinucleotide sequences. Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the group consisting of SEQ ID NOS:1-5, and contiguous regions thereof corresponding to the target sequence.

Additional embodiments provide a method for detecting a cell proliferative disorder of a prostate peripheral zone, or for distinguishing between a transitional and a peripheral zone of origin of a prostate cell proliferative disorder, comprising: obtaining a biological sample having subject genomic DNA; extracting the genomic DNA; treating the genomic DNA, or a fragment thereof, with one or more reagents to convert 5-position unmethylated cytosine bases to uracil or to another base that is detectably dissimilar to cytosine in terms of hybridization properties; contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a sequence selected from the group consisting of SEQ ID NOS:2-5, and complements thereof, wherein the genomic DNA or the fragment thereof is either amplified to produce an amplificate, or is not amplified; and determining, based on a presence or absence of, or on a property of said amplificate, the methylation state of at least one CpG dinucleotide sequence of SEQ ID NO:1, or an average, or a value reflecting an average methylation state of a plurality of CpG dinucleotide sequences of SEQ ID NO:1. Preferably, at least one such hybridizing nucleic acid molecule or peptide nucleic acid molecule is bound to a solid phase. Preferably, determining comprises use of at least two methods selected from the group consisting of: hybridizing at least one nucleic acid molecule comprising a contiguous sequence at least 9 nucleotides in length that is complementary to, or hybridizes under moderately stringent or stringent conditions to a sequence selected from the group consisting of SEQ ID NOS:2-5, and complements thereof, hybridizing at least one nucleic acid molecule, bound to a solid phase, comprising a contiguous sequence at least 9 nucleotides in length that is complementary to, or hybridizes under moderately stringent or stringent conditions to a sequence selected from the group consisting of SEQ ID NOS:2-5, and complements thereof; hybridizing at least one nucleic acid molecule comprising a contiguous sequence at least 9 nucleotides in length that is complementary to, or hybridizes under moderately stringent or stringent conditions to a sequence selected from the group consisting of SEQ ID NOS:2-5, and complements thereof, and extending at least one such hybridized nucleic acid molecule by at least one nucleotide base; and sequencing of the amplificate.

Further embodiments provide a method for detecting a cell proliferative disorder of a prostate peripheral zone, or for distinguishing between a transitional and a peripheral zone of origin of a prostate cell proliferative disorder, comprising: obtaining a biological sample having subject genomic DNA; extracting the genomic DNA; contacting the genomic DNA, or a fragment thereof, comprising SEQ ID NO:1 or a sequence that hybridizes under stringent conditions to SEQ ID NO:1, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence of SEQ ID NO:1, or an average, or a value reflecting an average methylation state of a plurality of CpG dinucleotide sequences of SEQ ID NO:1. Preferably, the digested or undigested genomic DNA is amplified prior to said determining.

Additional embodiments provide novel genomic and chemically modified nucleic acid sequences, as well as oligonucleotides and/or PNA-oligomers for analysis of cytosine methylation patterns within the GSTP1 promoter region (SEQ ID NO:1).

### BRIEF DESCRIPTION OF THE DRAWINGS

FIG. 1 shows the analysis of bisulfite treated GSTP1 promoter DNA using the MethyLight™ assay, performed according to EXAMPLE 1 herein below. The Y-axis shows the percentage of methylation at the CpG positions covered by the probes. The bar on the left hand side of the diagram illustrates the mean methylation levels of all samples originating from the prostate transitional zone, whereas the bar on the right hand side of the diagram illustrates the mean methylation levels of all samples originating from the prostate peripheral zone. The DNA of tumors originating in the peripheral zone is hypermethylated relative to that of tissue originating from the transitional zone.

### DETAILED DESCRIPTION OF THE INVENTION

Definitions:

The term “Observed/Expected Ratio” (“O/E Ratio”) refers to the frequency of CpG dinucleotides within a particular DNA sequence, and corresponds to the [number of CpG sites/(number of C bases×number of G bases)]×band length for each fragment.

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb in length.

The term “methylation state” or “methylation status” refers to the presence or absence of 5-methylcytosine (“5-mCyt”) at one or a plurality of CpG dinucleotides within a DNA sequence. Methylation states at one or more particular palindromic CpG methylation sites (each having two CpG CpG dinucleotide sequences) within a DNA sequence include “unmethylated,” “fully-methylated” and “hemi-methylated.” The term “hemi-methylation” or “hemimethylation” refers to the methylation state of a palindromic CpG methylation site, where only a single cytosine in one of the two CpG dinucleotide sequences of the palindromic CpG methylation site is methylated (e.g., 5′-CCMGG-3′ (top strand): 3′-GGCC-5′ (bottom strand)).

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “microarray” refers broadly to both “DNA microarrays,” and ‘DNA chip(s),’ as recognized in the art, encompasses all art-recognized solid supports, and encompasses all methods for affixing nucleic acid molecules thereto or synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analyzed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to a HeavyMethyl™ MethylLight™ assay, which is a variation of the MethylLight™ assay, wherein the MethylLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

The term “hybridization” is to be understood as a bond of an oligonucleotide to a complementary sequence along the lines of the Watson-Crick base pairings in the sample DNA, forming a duplex structure.

“Stringent hybridization conditions,” as defined herein, involve hybridizing at 68° C. in 5×SSC/5× Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

Overview:

According to the present invention, the methylation status of the promoter region and exons 1 and 2 of the GSTP1 gene (as disclosed in SEQ ID NO:1) was analyzed in a blinded protocol involving seventeen (17) samples of prostate adenocarcinoma from prostate cancer patients. Some were peripheral zone tumors and some were transitional zone tumors. For assay of methylation status, a real-time methylation-specific PCR was carried out upon bisulfite treated DNA using fluorescent labeled probes in a real-time PCR assay covering GSTP1 CpG positions of interest. The assay used (Jeronimo et al. J. Natl. Cancer Inst., 93:1747-52, 2001) is a variant of the Taqman™-based assay known as the MethyLight™ assay.

The present invention provides for molecular GSTP1 (Gluthione S-Transferase P gene; accession no. NM_{—}000852) markers that have novel utility for the analysis of methylation patterns within the promoter region and exons 1 and 2 of the GSTP1 gene. The markers are useful in novel methods to effectively distinguish among benign hyperplasia of the prostate, different grades of prostate cancer, and for the precise localization of the zone of origin of the cancer to provide sensitive, accurate and non-invasive methods for the diagnosis and/or prognosis of prostate cell proliferative disorders, including proliferative disorder of the peripheral zone of the prostate.

Bisulfite modification of DNA is an art-recognized tool used to assess CpG methylation status. 5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing, because 5-methylcytosine has the same base pairing behavior as cytosine. Moreover, the epigenetic information carried by 5-methylcytosine is completely lost during, e.g., PCR amplification.

The most frequently used method for analyzing DNA for the presence of 5-methylcytosine is based upon the specific reaction of bisulfite with cytosine whereby, upon subsequent alkaline hydrolysis, cytosine is converted to uracil which corresponds to thymine in its base pairing behavior. Significantly, however, 5-methylcytosine remains unmodified under these conditions. Consequently, the original DNA is converted in such a manner that methylcytosine, which originally could not be distinguished from cytosine by its hybridization behavior, can now be detected as the only remaining cytosine using standard, art-recognized molecular biological techniques, for example, by amplification and hybridization, or by sequencing. All of these techniques are based on differential base pairing properties, which can now be fully exploited.

The prior art, in terms of sensitivity, is defined by a method comprising enclosing the DNA to be analyzed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996). It is thus possible to analyze individual cells for methylation status, illustrating the utility and sensitivity of the method. Currently, however, only individual regions of a length of up to approximately 3000 base pairs are analyzed, and a global analysis of cells for thousands of possible methylation events is not feasible. Moreover, this agarose-matrix method cannot reliably analyze very small fragments from small sample quantities. Such fragments are lost, despite the diffusion-resisting properties of the matrix. An overview of art-recognized methods for detecting 5-methylcytosine is provided by Rein, T., et al., Nucleic Acids Res., 26:2255, 1998.

The bisulfite technique, barring few exceptions (e.g., Zeschnigk M, et al., Eur J Hum Genet. 5:94-98, 1997), is currently only used in research. In all instances, short, specific fragments of a known gene are amplified subsequent to a bisulfite treatment, and either completely sequenced (Olek & Walter, Nat Genet. 1997 17:275-6, 1997), subjected to one or more primer extension reactions (Gonzalgo & Jones, Nucleic Acids Res., 25:2529-31, 1997; WO 95/00669; U.S. Pat. No. 6,251,594) to analyze individual cytosine positions, or treated by enzymatic digestion (Xiong & Laird, Nucleic Acids Res., 25:2532-4, 1997). Detection by hybridization has also been described in the art (Olek et al., WO 99/28498). Additionally, use of the bisulfite technique for methylation detection with respect to individual genes has been described (Grigg & Clark, Bioessays, 16:431-6, 1994; Zeschnigk M, et al., Hum Mol Genet., 6:387-95, 1997; Feil R, et al., Nucleic Acids Res., 22:695-, 1994; Martin V, et al., Gene, 157:261-4, 1995; WO 9746705 and WO 9515373).

The present invention provides for the use of the bisulfite technique, in combination with one or more methylation assays, for determination of the methylation status of CpG dinuclotide sequences within the GSTP1 promoter regions. According to the present invention, determination of the methylation status of GSTP1 CpG dinucleotide sequences has diagnostic and prognostic utility.

Methylation Assay Procedures. Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, use of methylation-sensitive restriction enzymes, etc.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

COBRA. COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the interested CpG islands, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Preferably, assays such as “MethyLight™” (a fluorescence-based real-time PCR technique) (Eads et al., Cancer Res. 59:2302-2306, 1999), Ms-SNuPE (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with other of these methods.

MethyLight™. The MethyLight™ assay is a high-throughput quantitative methylation assay that utilizes fluorescence-based real-time PCR (TaqMan®) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight™ assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight™ process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific gene (or methylation-altered DNA sequence or CpG island); TaqMan® probes; optimized PCR buffers and deoxynucleotides; and Taq polymerase.

Ms-SNuPE. The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997).

Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest.

Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

MSP. MSP (methylation-specific PCR) allows for assessing the methylation status of virtually any group of CpG sites within a CpG island, independent of the use of methylation-sensitive restriction enzymes (Herman et al. Proc. Nat. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus umnethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

MCA. The MCA technique is a method that can be used to screen for altered methylation patterns in genomic DNA, and to isolate specific sequences associated with these changes (Toyota et al., Cancer Res. 59:2307-12, 1999). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

GSTP1 PROMOTER CpG DINUCLEOTIDE SEQUENCES WERE DETERMINED TO HAVE UTILITY FOR THE DETECTION OF A CELL PROLIFERATIVE DISORDER OF

THE PERIPHERAL ZONE OF THE PROSTATE, AND FOR DISTINGUISHING CELL PROLIFERATIVE DISORDERS ORIGINATING IN THE TRANSITIONAL ZONE FROM

THOSE ORIGINATING IN THE PERIPHERAL ZONE OF THE PROSTATE

The present invention is based upon the analysis of methylation levels within the promoter region and exons 1 and 2 of the GSTP1 gene (SEQ ID NO:1), said region of the genome being well characterised in terms of both sequence and function. Hypermethylation of this region has been associated with the development of metastatic prostate cell proliferative disorders (summarized herein above, under “Background”).

Particular embodiments of the present invention provide a novel application of the analysis of methylation levels and/or patterns within SEQ ID NO:1 that enables the precise localisation of the zone of origin of prostatic cell proliferative disorders. Identification of the zone of origin of the disorder is directly linked with disease prognosis, and the disclosed method thereby enables the physician and patient to make better and more informed treatment decisions. According to the present invention, the development of a molecular marker for the precise localization of the zone of origin of a prostatic cell proliferative disorder enables a non-invasive and more accurate method as opposed to currently used subjective and invasive methods such as biopsy and transrectal ultrasound.

The present invention provides novel uses for analysis of the methylation levels within the GSTP1 promoter region and exons 1 and 2 (SEQ ID NO:1). Additional embodiments provide genomic and chemically modified nucleic acid sequences, as well as oligonucleotides and/or PNA-oligomers for analysis of cytosine methylation patterns within said region.

An objective of the invention comprises analysis of the methylation state of the CpG dinucleotides within the genomic sequence according to SEQ ID NO:1 and sequences complementary thereto. SEQ ID NO:1 corresponds to the core promoter region and exons 1 and 2 of the human GSTP1 gene.

In a preferred embodiment of the method, the objective comprises analysis of a chemically modified nucleic acid comprising a sequence of at least 18 nucleotide bases in length, according to one of SEQ ID NO:2 to SEQ ID NO:5 and sequences complementary thereto. The sequences of SEQ ID NOS: 2-5 provide chemically modified versions of the nucleic acid according to SEQ ID NO:1, wherein the chemical modification of said sequence results in the synthesis of a nucleic acid having a sequence that is unique and distinct from SEQ ID NO:1 as follows (see also the following TABLE 1): SEQ ID NO:1, sense DNA strand of GSTP1 core promoter sequence plus exons 1 and 2; SEQ ID NO:2, chemically converted SEQ ID NO:1, wherein “C” →“T,” but “CpG” remains “CpG” (i.e., corresponds to case where, for SEQ ID NO:1, all “C” residues of CpG dinucleotide sequences are methylated and are thus not converted); SEQ ID NO:3, complement of SEQ ID NO:1, wherein “C” →“T,” but “CpG” remains “CpG” (i.e., corresponds to case where, for the complement (antisense strand) of SEQ ID NO:1, all “C” residues of CpG dinucleotide sequences are methylated and are thus not converted); SEQ ID NO:4, chemically converted SEQ ID NO:1, wherein “C” →“T” for all “C” residues, including those of “CpG” dinucleotide sequences (i.e., corresponds to case where, for SEQ ID NO:1, all “C” residues of CpG dinucleotide sequences are unmethylated); SEQ ID NO:5, complement of SEQ ID NO:1, wherein “C” →“T” for all “C” residues, including those of “CpG” dinucleotide sequences (i.e., corresponds to case where, for the complement (antisense strand) of SEQ ID NO:1, all “C” residues of CpG dinucleotide sequences are umnethylated).

TABLE 1

Description of SEQ ID NOS: 1-5

| | Relationship to | |
|---|---|---|
| SEQ ID NO | SEQ ID NO:1 | Nature of cytosine base conversion |

| | | |
|---|---|---|
| SEQ ID NO:1 | Sense strand (GSTP1 core | None; untreated sequence |
| | promoter plus exons 1 and 2) | |
| SEQ ID NO:2 | Chemically-treated sense | “C” → “T,” but “CpG” remains “CpG” (all |
| | strand | “C” residues of CpGs are methylated) |
| SEQ ID NO:3 | Chemically-treated antisense | “C” → “T,” but “CpG” remains “CpG” (all |
| | strand | “C” residues of CpGs are methylated) |
| SEQ ID NO:4 | Chemically-treated sense | “C” → “T” for all “C” residues (all “C” |
| | strand | residues of CpGs are unmethylated) |
| SEQ ID NO:5 | Chemically-treated antisense | “C” → “T” for all “C” residues (all “C” |
| | strand | residues of CpGs are unmethylated) |

Significantly, heretofore, the nucleic acid sequences and molecules according to SEQ ID NO:1 to SEQ ID NO:5 were not implicated in or connected with the ascertainment of the zone of origin of prostate cell proliferative disorders.

In an alternative preferred embodiment, such analysis comprises the use of an oligonucleotide or oligomer for detecting the cytosine methylation state within genomic or pretreated (chemically modified) DNA, according to SEQ ID NO:1 to SEQ ID NO:5. Said oligonucleotide or oligomer comprising a nucleic acid sequence having a length of at least nine (9) nucleotides which hybridizes, under moderately stringent or stringent conditions (as defined herein above), to a pretreated nucleic acid sequence according to SEQ ID NO:2 to SEQ ID NO:5 and/or sequences complementary thereto, or to a genomic sequence comprising SEQ ID NO:1 and/or sequences complementary thereto.

Thus, the present invention includes nucleic acid molecules (e.g., oligonucleotides and peptide nucleic acid (PNA) molecules (PNA-oligomers)) that hybridize under moderately stringent and/or stringent hybridization conditions to all or a portion of the sequences of SEQ ID NOS:1-5, or to the complements thereof. The hybridizing portion of the hybridizing nucleic acids is typically at least 10, 15, 20, 25, 30 or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NOS:1-5, or to the complements thereof.

Hybridizing nucleic acids of the type described herein can be used, for example, as a primer (e.g., a PCR primer), or a diagnostic and/or prognostic probe or primer. Preferably, hybridization of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO:1 (such as GSTP1 allelic variants and SNPs), rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NO:1, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:

n to (n+(X−1));

where n=1, 2, 3, . . . (Y−(X−1));

where Y equals the length (nucleotides or base pairs) of SEQ ID NO:1 (2,785);

where X equals the common length (in nucleotides) of each oligonucleotide in the set (e.g., X=20 for a set of consecutively overlapping 20-mers); and

where the number (Z) of consecutively overlapping oligomers of length X for a given SEQ ID NO of length Y is equal to Y−(X−1). For example Z=2,785-19=2,766 for either sense or antisense sets of SEQ ID NO:1, where X=20.

Preferably, the set is limited to those oligomers that comprise at least one CpG, TpG or CpA dinucleotide.

Examples of inventive 20-mer oligonucleotides include the following set of 2,766 oligomers (and the antisense set complementary thereto), indicated by polynucleotide positions with reference to SEQ ID NO:1 (GSTP1):

1-20, 2-21, 3-22, 4-23, 5-24 . . . 2764-2783, 2765-2784 and 2766-2785.

Preferably, the set is limited to those oligomers that comprise at least one CpG, TpG or CpA dinucleotide.

Likewise, examples of 25-mer oligonucleotides include the following set of 2,761 oligomers (and the antisense set complementary thereto), indicated by polynucleotide positions with reference to SEQ ID NO:1:

1-25, 2-26, 3-27, 4-28, 5-29 . . . 2759-2783, 2760-2784 and 2761-2785.

Preferably, the set is limited to those oligomers that comprise at least one CpG, TpG or CpA dinucleotide.

The present invention encompasses, for each of SEQ ID NOS:1-5 (sense and antisense), multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequence corresponding to SEQ ID NO:1. Preferred sets of such oligonucleotides or modified oligonucleotides of length X are those consecutively overlapping sets of oligomers corresponding to SEQ ID NOS:1-5 (and to the complements thereof). Preferably, said oligomers comprise at least one CpG, TpG or CpA dinucleotide. Included in these preferred sets are the preferred oligomers corresponding to SEQ ID NOS:6-8.

Particularly preferred oligonucleotides or oligomers according to the present invention are those in which the cytosine of the CpG dinucleotide (or of the corresponding converted TpG or CpA dinculeotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc.

The oligonucleotide may also comprise at least one art-recognized modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of each of the CpG dinucleotides of genomic sequence SEQ ID NO:1 and sequences complementary thereto, or to the corresponding CpG, TpG or CpA dinucleotide within a sequence of the pretreated nucleic acids according to SEQ ID NO:2 to SEQ ID NO:5 and sequences complementary thereto. However, it is anticipated that for economic or other factors it may be preferable to analyze a limited selection of the CpG dinucleotides within said sequences, and the content of the set of oligonucleotides is altered accordingly.

Therefore, in particular embodiments, the present invention provides a set of at least four (4) (oligonucleotides and/or PNA-oligomers) useful for detecting the cytosine methylation state in pretreated genomic DNA (SEQ ID NO:2 to SEQ ID NO:5 and sequences complementary thereto), or in genomic DNA (SEQ ID NO:1 and sequences complementary thereto). These probes enable diagnosis, prognosis, and/or therapy of genetic and epigenetic parameters of cell proliferative disorders. The set of oligomers may also be used for detecting single nucleotide polymorphisms (SNPs) in pretreated genomic DNA (SEQ ID NO:2 to SEQ ID NO:5, and sequences complementary thereto), or in genomic DNA (SEQ ID NO:1, and sequences complementary thereto).

In preferred embodiments, at least one, and more preferably all members of a set of oligonucleotides is bound to a solid phase.

In further embodiments, the present invention provides a set of at least two (2) oligonucleotides that are used as ‘primer’ oligonucleotides for amplifying DNA sequences of one of SEQ ID NO:1 to SEQ ID NO:5 and sequences complementary thereto, or segments thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may be composed of silicon, glass, polystyrene, aluminum, steel, iron, copper, nickel, silver, or gold. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example, via a confocal microscope. Cy3 and Cy5 dyes, besides many others, are commercially available.

The present invention further provides a method for ascertaining genetic and/or epigenetic parameters of the promoter region and exons 1 and 2 of the GSTP1 gene according to SEQ ID NO:1 within a subject by analyzing cytosine methylation and single nucleotide polymorphisms. Said method comprising contacting a nucleic acid comprising SEQ ID NO:1 in a biological sample obtained from said subject with at least one reagent or a series of reagents, wherein said reagent or series of reagents, distinguishes between methylated and non-methylated CpG dinucleotides within the target nucleic acid.

Preferably, said method comprises the following steps: In the first step, a sample of the tissue to be analysed is obtained. The source may be any suitable source, such as cell lines, histological slides, biopsies, tissue embedded in paraffin, bodily fluids, ejaculate, urine, blood and all possible combinations thereof.

In the second step, DNA is isolated from the sample. Extraction may be by means that are standard to one skilled in the art, including the use of detergent lysates, sonification and vortexing with glass beads. Once the nucleic acids have been extracted, the genomic double stranded DNA is used in the analysis.

In the third step of the method, the genomic DNA sample is treated in such a manner that cytosine bases which are unmethylated at the 5′-position are converted to uracil, thymine, or another base which is dissimilar to cytosine in terms of hybridization behavior. This will be understood as ‘pretreatment’ herein.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior.

In the fourth step of the method, fragments of the pretreated DNA are amplified, using sets of primer oligonucleotides according to the present invention, and a preferably heat-stable polymerase. The amplification of several DNA segments can be carried out simultaneously in one and the same reaction vessel. Typically, the amplification is carried out using a polymerase chain reaction (PCR). The set of primer oligonucleotides includes at least two oligonucleotides whose sequences are each reverse complementary, identical, or hybridize under stringent or highly stringent conditions to an at least 18-base-pair long segment of the base sequences of SEQ ID NO:2 to SEQ ID NO:5 and sequences complementary thereto.

In an alternate embodiment of the method, the methylation status of preselected CpG positions within the nucleic acid sequences comprising SEQ ID NO:2 to SEQ ID NO:5 may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer which hybridizes to a bisulfite treated CpG dinucleotide. Therefore, the sequence of said primers comprises at least one CpG, TpG or CpA dinucleotide. MSP primers specific for non-methylated DNA contain a “T” at the 3′ position of the C position in the CpG. Preferably, therefore, the base sequence of said primers is required to comprise a sequence having a length of at least 9 nucleotides which hybridizes to a pretreated nucleic acid sequence according to SEQ ID NO:2 to SEQ ID NO:5 and sequences complementary thereto, wherein the base sequence of said oligomers comprises at least one CpG, TpG or CpA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labeled amplificates have a single positive or negative net charge, allowing for better detectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapour phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionization process via the matrix is considerably less efficient. In MALDI-TOF spectrometry, the selection of the matrix plays an eminently important role. For the desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In the fifth step of the method, the amplificates obtained during the fourth step of the method are analysed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer.

Amplificates obtained by means of both standard and methylation specific PCR may be further analyzed by means of hybridization-based methods such as, but not limited to, array technology and probe based technologies as well as by means of techniques such as sequencing and template directed extension.

In one embodiment of the method, the amplificates synthesised in step four are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; said oligonucleotides contain at least one base sequence having a length of at least 9 nucleotides which is reverse complementary or identical to a segment of the base sequences specified in the present Sequence Listing; and the segment comprises at least one CpG, TpG or CpA dinucleotide.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within the sequence according to SEQ ID NO:1, and the equivalent positions within SEQ ID NOS:2 to 5. Said oligonucleotides may also be present in the form of peptide nucleic acids. The non-hybridized amplificates are then removed.

In the final step of the method, the hybridized amplificates are detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes that are hybridised to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which is designed to hybridize to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethylLight™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulfite treated DNA, and moreover for methylation analysis within CpG dinucleotides.

A further suitable method for the use of probe oligonucleotides for the assessment of methylation by analysis of bisulfite treated nucleic acids comprises the use of blocker oligonucleotides. The use of such blocker oligonucleotides has been described by Yu et al., BioTechniques 23:714-720, 1997. Blocking probe oligonucleotides are hybridized to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpG’ at the position in question, as opposed to a ‘CpA.’.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivitized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase. In a further preferred embodiment of the method, the fifth step of the method comprises the use of template-directed oligonucleotide extension, such as MS-SNuPE as described by Gonzalgo & Jones, Nucleic Acids Res. 25:2529-2531, 1997.

In yet a further embodiment of the method, the fifth step of the method comprises sequencing and subsequent sequence analysis of the amplificate generated in the third step of the method (Sanger F., et al., Proc Natl Acad Sci USA 74:5463-5467, 1977).

Additional embodiments of the invention provide a method for the analysis of the methylation status of genomic DNA according to the invention (SEQ ID NO:1) without the need for pretreatment.

In the first step of such additional embodiments, the genomic DNA sample is isolated from tissue or cellular sources. Preferably, such sources include cell lines, histological slides, body fluids, or tissue embedded in paraffin. Extraction may be by means that are standard to one skilled in the art, including but not limited to the use of detergent lysates, sonification and vortexing with glass beads. Once the nucleic acids have been extracted, the genomic double-stranded DNA is used in the analysis.

In a preferred embodiment, the DNA may be cleaved prior to the treatment, and this may be by any means standard in the state of the art, in particular with methylation-sensitive restriction endonucleases.

In the second step, the DNA is then digested with one or more methylation sensitive restriction enzymes. The digestion is carried out such that hydrolysis of the DNA at the restriction site is informative of the methylation status of a specific CpG dinucleotide.

In the third step, which is optional but a preferred embodiment, the restriction fragments are amplified. This is preferably carried out using a polymerase chain reaction, and said amplificates may carry suitable detectable labels as discussed above, namely fluorophore labels, radionuclides and mass labels.

In the final step the amplificates are detected. The detection may be by any means standard in the art, for example, but not limited to, gel electrophoresis analysis, hybridization analysis, incorporation of detectable tags within the PCR products, DNA array analysis, MALDI or ESI analysis.

Diagnostic and/or Prognostic Assays for Cancer and Hyperproliferative Disorders

The present invention enables diagnosis and/or prognosis of events which are disadvantageous to patients or individuals in which important genetic and/or epigenetic parameters within the GSTP1 promoter may be used as markers. Said parameters obtained by means of the present invention may be compared to another set of genetic and/or epigenetic parameters, the differences serving as the basis for a diagnosis and/or prognosis of events which are disadvantageous to patients or individuals.

Specifically, the present invention provides for diagnostic and/or prognostic cancer assays based on measurement of differential methylation of GSTP1 CpG dinucleotide sequences. Preferred gene sequences useful to measure such differential methylation are represented herein by SEQ ID NOS:1-5. Typically, such assays involve obtaining a tissue sample from a test tissue, performing an assay to measure the methylation status of at least one of the inventive GSTP1-specific CpG dinucleotide sequences derived from the tissue sample, relative to a control sample, and making a diagnosis or prognosis based thereon.

In particular preferred embodiments, inventive oligomers are used to assess GSTP1-specific CpG dinucleotide methylation status, such as those based on SEQ ID NOS:1-5, including the representative preferred oligomers corresponding to SEQ ID NOS:6-8, or arrays thereof, as well as a kit based thereon are useful for the diagnosis and/or prognosis of cancer and/or other prostate cell proliferative disorders.

The present invention moreover relates to a method for manufacturing a diagnostic agent and/or therapeutic agent for the diagnosis and/or therapy cancer, the diagnostic agent and/or therapeutic agent being characterized in that at least one primer or probe based on SEQ ID NOS:1-5 is used for manufacturing it, possibly together with suitable additives and ancillary agents.

Kits

Moreover, an additional aspect of the present invention is a kit comprising, for example: a bisulfite-containing reagent; a set of primer oligonucleotides containing at least two oligonucleotides whose sequences in each case correspond, are complementary, or hybridize under stringent or highly stringent conditions to a 18-base long segment of the sequences SEQ ID NO:1-5; oligonucleotides and/or PNA-oligomers; as well as instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethyLight™, HeavyMethyl™, COBRA, and nucleic acid sequencing. However, a kit along the lines of the present invention can also contain only part of the aforementioned components.

While the present invention has been described with specificity in accordance with certain of its preferred embodiments, the following example serves only to illustrate the invention and is not intended to limit the invention within the principles and scope of the broadest interpretations and equivalent configurations thereof.

### EXAMPLE 1

### (Measurement of GSTP1 Promoter-Specific CpG Methylation Status has Novel Utility for Diagnosis and Prognosis of Prostate Cellular Disorders)

In the following example the methylation status of the promoter region and exons 1 and 2 of the GSTP1 gene (as disclosed in SEQ ID NO:1) was analyzed in seventeen (17) samples of prostate adenocarcinoma from prostate cancer patients. Some were peripheral zone tumors and some were transitional zone tumors. The samples were processed in a blinded protocol (trial). The MethyLight™ assay procedure, a real-time methylation-specific Taqman™-based PCR assay, along with bisulfite treated DNA and fluorescent-labeled oligonucleotide hybridization probes, was used to determine methylation status of GSTP1 CpG positions of interest. The MethyLight™ assay herein differs from the previously published MSP-based GSTP1 assay (Jeronimo et al. J. Natl. Cancer Inst., 93:1747-52, 2001) in that the present labeled hybridization probes are designed to cover (i.e., be complementary to) CpG dicucleotide positions of interest (i.e., CpG specific probes, in contrast to the CpG-specific primer(s) used in MSP).

Methods. DNA was extracted from the samples using a Qiagen™ extraction kit. The DNA from each sample was treated using a bisulfite solution (hydrogen sulfite, disulfite) according to the agarose bead method (Olek et al. Nucleic Acids Res. 24:5064-6, 1996). The treatment is such that all non-methylated cytosines within the sample are converted to thymine. Conversely, 5-methylated cytosines within the sample remain unmodified.

The methylation status was determined with a MethyLight™ assay designed for the CpG island of interest and a control fragment from the beta-actin gene (Eads et al. Cancer Res. 61:3410-8, 2001). The CpG island assay covers CpG sites in both the primers and the taqman-style probe, while the control gene does not. The control reaction was used to normalize the levels of input DNA, and this reaction amplifies all DNA regardless of the methylation state. The control gene is used as a measure of total DNA concentration, and the CpG island assay determines the methylation levels at that site. The GSTP1 CpG island assay was performed using the following primers and probes:

| | | |
|---|---|---|
| Primer: | AGTTGCGCGGCGATTTC; | (SEQ ID NO:6) |
| Primer: | GCCCCAATACTAAATCACGACG; and | (SEQ ID NO:7) |
| Probe: | CGAATCTCTCGAACGATCGCATCCA. | (SEQ ID NO:8) |

The corresponding control assay was performed using the following primers and probes:

| | | |
|---|---|---|
| Primer: | TGGTGATGGAGGAGGTTTAGTAAGT; | (SEQ ID NO:9) |
| Primer: | AACCAATAAAACCTACTCCTCCCTTAA; and | (SEQ ID NO:10) |
| Probe: | ACCACCACCCAACACACAATAACAAACACA. | (SEQ ID NO:11) |

The reactions were run in triplicate on each DNA sample with the following assay conditions:

Reaction solution: (500 nM primers; 2,500 nM probe; 3.5 mM magnesium chloride; 1 unit of taq polymerase; 200 μM dNTPs; 10 μl of DNA, in a final reaction volume of 20 μl);

Cycling conditions: (95° C. for 15 seconds; 60° C. for 1 minute) (50 cycles).

The data was analyzed using a PMR calculation previously described in the literature (Eads et al. Cancer Res. 61:3410-8, 2001). The ratio of amplification with the GSTP1 methylation assay to amplification with the total DNA control assay was used to calculate the methylation level. The samples were sorted into three groups based on their methylation level: high methylation, low methylation, and no methylation. For two of the samples, the control assay indicated that there was insufficient DNA for methylation analysis. The results for the 17 samples are given in the following TABLE 1:

TABLE 1
Differential methylation of GSTP1 CpG dinucleotide
sequences between transitional zone and
peripheral zone prostate tumors (blinded protocol)

| Sample ID | Methylation Level | Methylation Call |
|---|---|---|
| 100507972 | 1.235 | High methylation |
| 100508045 | 0.8411 | High methylation |
| 100507980 | 0.4600 | High methylation |
| 100508003 | 0.3220 | High methylation |
| 100508037 | 0.1069 | High methylation |
| 100508079 | 0.0649 | Low methylation |
| 100507998 | 0.0561 | Low methylation |
| 100507964 | 0.0307 | Low methylation |
| 100508061 | 0.0120 | Low methylation |
| 100507930 | 0.0095 | Low methylation |
| 100508053 | 0 | No methylation |
| 100508029 | 0 | No methylation |
| 100507948 | 0 | No methylation |
| 100507899 | 0 | No methylation |
| 100508087 | 0 | No methylation |
| 100508011 | N.D. | Insufficient DNA |
| 100507956 | N.D. | Insufficient DNA |

When the samples were unblinded, four of the five highly methylated samples were peripheral zone cancers, and four of the five samples with no methylation were transitional zone cancers. The samples with low methylation were a mixture of the two sample types.

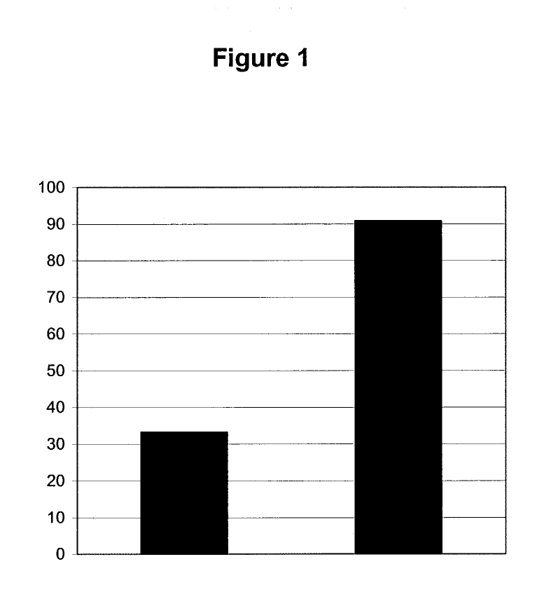

FIG. 1 graphically shows the methylation data for the 17 tissue samples analyzed in theis EXAMPLE 1. The Y-axis shows the percentage of methylation at the CpG positions covered by the probes. The bar on the left hand side of the diagram illustrates the mean methylation levels of all samples originating from the prostate transitional zone, whereas the bar on the right hand side of the diagram illustrates the mean methylation levels of all samples originating from the prostate peripheral zone. The DNA of tumors originating in the peripheral zone is hypermethylated relative to that of tissue originating from the transitional zone.

These data show, according to the present invention, that peripheral zone tumors are readily distinguishable from transitional zone tumors, based on differential GSTP1 methylation status.

Furthermore, the methods according to the present invention are, relative to those of the art, accurate and non-invasive. To practice the methods disclosed and enabled herein it is necessary to obtain a biological sample comprising genomic DNA corresponding to a test subject. However, the scope of the biological sample having utility according to the present invention, is broad and encompasses non-invasive (e.g., bodily fluids), as well as traditional art-recognized invasive means (e.g., biopsy and transrectal ultrasound).

Preferably, subject biological samples of the present invention are selected from the subject sample group consisting of cell lines, histological slides, biopsies, paraffin-embedded tissue, bodily fluids, ejaculate, urine, blood, and combinations thereof.

Preferably, subject biological samples are obtained by non-invasive procedures including, but not limited to collection of subject bodily fluids, ejaculate, urine, blood, and combinations thereof.

Therefore, the present invention provides for molecular GSTP1 markers that have novel, specific, credible and substantial utility in effectively distinguishing among benign hyperplasia of the prostate and different grades of prostate cancer. Additionally, the present invention provides for molecular GSTP1 markers that have such utility for the precise localization of the zone of origin to provide sensitive, accurate and non-invasive methods for the diagnosis, prognosis and treatment of prostate cell proliferative disorders.

We claim:

1. A method for detecting a cell proliferative disorder of a prostate peripheral zone, or for distinguishing between a transitional and a peripheral zone of origin of a prostate cell proliferative disorder, comprising:
a) obtaining, from a subject, a biological sample having subject genomic DNA;
b) contacting the genomic DNA, or a fragment thereof, with one reagent or a plurality of reagents sufficient for distinguishing between methylated and non methylated CpG dinucleotide sequences within a target sequence of the genomic DNA, or fragment thereof, wherein the target sequence comprises, or hybridizes under stringent conditions to, at least 18 contiguous nucleotides of SEQ ID NO:1, said contiguous nucleotides comprising at least one CpG dinucleotide sequence; and
c) determining, based at least in part on said distinguishing, the methylation state of at least one target CpG dinucleotide sequence, or an average, or a value reflecting an average methylation state of a plurality of target CpG dinucleotide sequences, whereby at least one of detecting the prostate cell proliferative disorder, or distinguishing between a transitional and a peripheral zone of origin of the prostate cell proliferative disorder is, at least in part, afforded.

2. The method of claim 1, wherein distinguishing between methylated and non methylated CpG dinucleotide sequences within the target sequence comprises converting unmethylated cytosine bases within the target sequence to uracil or another base that is detectably dissimilar to cytosine in terms of hybridization properties.

3. The method of claim 1, wherein distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the group consisting of SEQ ID NOS:1-5, and contiguous regions thereof corresponding to the target sequence.

4. The method of claim 1, wherein the minimal length of contiguous nucleotides of SEQ ID NO:1 is selected from the group consisting of at least 18, 20, 25, 50, 100, 200, 500, 1000 and 2,785 contiguous nucleotides.

5. The method of claim 1, wherein the biological sample obtained from the subject is selected from the group consisting of cell lines, histological slides, biopsies, paraffin-embedded tissue, bodily fluids, ejaculate, urine, blood, and combinations thereof.

6. The method of claim 1, wherein distinguishing between methylated and non methylated CpG dinucleotide sequences within the target sequence comprises use of at least one nucleic acid molecule or peptide nucleic acid (PNA) molecule comprising, in each case a contiguous sequence at least 9 nucleotides in length that is complementary to, or hybridizes under moderately stringent or stringent conditions to a sequence selected from the group consisting of SEQ ID NOS:1-5, and complements thereof.

7. The method of claim 6, wherein the nucleic acid molecule or peptide nucleic acid (PNA) molecule, comprises a contiguous sequence at least 18 nucleotides in length that is complementary to, or hybridizes under moderately stringent or stringent conditions to a sequence selected from the group consisting of SEQ ID NOS:1-5, and complements thereof.

8. The method of claim 6, wherein the contiguous sequence comprises at least one CpG, TpG or CpA dinucleotide sequence.

9. The method of claim 8, wherein the first position of the dinucleotide sequence, in each case, is located at about the middle third of the contiguous sequence.

10. The method of claim 6, comprising use of at least two such nucleic acid molecules, or peptide nucleic acid (PNA) molecules.

11. The method of claim 6, comprising use of a set of such nucleic acid molecules, or peptide nucleic acid (PNA) molecules sufficient for determining the methylation state of all CpG dinucleotide sequences within SEQ ID NO:1 and sequences complementary thereto.

12. The method of claim 6, comprising use of at least two such nucleic acid molecules, or peptide nucleic acid (PNA) molecules as primer oligonucleotides for the amplification of a sequences selected from the group consisting of SEQ ID NOS:1-5, sequences complementary thereto, and regions thereof that comprise, or hybridize under stringent conditions to the primers.

13. The method of claim 10, wherein at least one such nucleic acid molecule, or peptide nucleic acid (PNA) molecule is bound to a solid phase.

14. The method of claim 6, comprising use of at least four such nucleic acid molecules, or peptide nucleic acid (PNA) molecules.

15. A method for detecting a cell proliferative disorder of a prostate peripheral zone, or for distinguishing between a transitional and a peripheral zone of origin of a prostate cell proliferative disorder, comprising:
a) obtaining, from a subject, a biological sample having subject genomic DNA;
b) extracting the genomic DNA;
c) treating the genomic DNA, or a fragment thereof, with one or more reagents to convert 5-position unmethylated cytosine bases to uracil or to another base that is detectably dissimilar to cytosine in terms of hybridization properties;
d) contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a sequence selected from the group consisting of SEQ ID NOS:2-5, and complements thereof, wherein the genomic DNA or the fragment thereof is either amplified to produce an amplificate, or is not amplified; and
e) determining, based on a presence or absence of, or on a property of said amplificate, the methylation state of at least one CpG dinucleotide sequence of SEQ ID NO:1, or an average, or a value reflecting an average methylation state of a plurality of CpG dinucleotide sequences of SEQ ID NO:1, whereby at least one of detecting the prostate cell proliferative disorder, or distinguishing between a transitional and a peripheral zone of origin of the prostate cell proliferative disorder is, at least in part, afforded.

16. The method of claim 15, wherein determining in step e), comprises hybridization of at least one nucleic acid molecule or peptide nucleic acid molecule in each case comprising a contiguous sequence at least 9 nucleotides in length that is complementary to, or hybridizes under moderately stringent or stringent conditions to a sequence selected from the group consisting of SEQ ID NOS:1-5, and complements thereof.

17. The method of claim 16, wherein at least one such hybridizing nucleic acid molecule or peptide nucleic acid molecule is bound to a solid phase.

18. The method of claim 16, wherein a plurality of such hybridizing nucleic acid molecules or peptide nucleic acid molecules are bound to a solid phase in the form of a nucleic acid or peptide nucleic acid array selected from the array group consisting of linear, hexagonal, rectangular, and combinations thereof.

19. The method of claim 15, wherein determining in step e), comprises: hybridizing at least one nucleic acid molecule comprising a contiguous sequence at least 9 nucleotides in length that is complementary to, or hybridizes under moderately stringent or stringent conditions to a sequence selected from the group consisting of SEQ ID NOS:2-5, and complements thereof; and extending at least one such hybridized nucleic acid molecule by at least one nucleotide base.

20. The method of claim 15, wherein determining in step e), comprises sequencing of the amplificate.

21. The method of claim 15, wherein contacting or amplifying in step d), comprises use of methylation-specific primers.

22. The method of claim 15, wherein determining in step e), comprises use of at least two methods selected from the group consisting of: hybridizing at least one nucleic acid molecule comprising a contiguous sequence at least 9 nucleotides in length that is complementary to, or hybridizes under moderately stringent or stringent conditions to a sequence selected from the group consisting of SEQ ID NOS:2-5, and complements thereof; hybridizing at least one nucleic acid molecule, bound to a solid phase, comprising a contiguous sequence at least 9 nucleotides in length that is complementary to, or hybridizes under moderately stringent or stringent conditions to a sequence selected from the group consisting of SEQ ID NOS:2-5, and complements thereof; hybridizing at least one nucleic acid molecule comprising a contiguous sequence at least 9 nucleotides in length that is complementary to, or hybridizes under moderately stringent or stringent conditions to a sequence selected from the group consisting of SEQ ID NOS:2-5, and complements thereof, and extending at least one such hybridized nucleic acid molecule by at least one nucleotide base; and sequencing of the amplificate.

23. The method of claim 15, wherein treating the genomic DNA, or the fragment thereof in step c), comprises use of a solution selected from the solution group consisting of bisulfite, hydrogen sulfite, disulfite, and combinations thereof.

24. The method of claim 15, wherein contacting or amplifying in step d) comprises use of at least one method selected from the group consisting of: use of a heat-resistant DNA polymerase as the amplification enzyme; use of a polymerase lacking 5′-3′ exonuclease activity; use of a polymerase chain reaction (PCR); generation of a amplificate nucleic acid molecule carrying a detectable labels; and combinations thereof.

25. The method of claim 24, wherein the detectable amplificate label is selected from the label group consisting of: fluorescent labels; radionuclides or radiolabels; amplificate mass labels detectable in a mass spectrometer; detachable amplificate fragment mass labels detectable in a mass spectrometer; amplificate, and detachable amplificate fragment mass labels having a single-positive or single-negative net charge detectable in a mass spectrometer; and combinations thereof.

26. The method of claim 25, comprising use of mass spectrometry for detecting amplificate, or detachable amplificate fragment mass labels.

27. The method of claim 26, wherein the mass spectrometry is selected from the group consisting of matrix assisted laser desorption/ionization mass spectrometry (MALDI), electron spray mass spectrometry (ESI), and combinations thereof.

28. The method of claim 15, wherein the biological sample obtained from the subject is selected from the group consisting of cell lines, histological slides, biopsies, paraffin-embedded tissue, bodily fluids, ejaculate, urine, blood, and combinations thereof.

29. The method of claim 15, further comprising in step d) the use of at least one nucleic acid molecule or peptide nucleic acid molecule comprising in each case a contiguous sequence at least 9 nucleotides in length that is complementary to, or hybridizes under moderately stringent or stringent conditions to a sequence selected from the group consisting of SEQ ID NOS:2-5, and complements thereof, wherein said nucleic acid molecule or peptide nucleic acid molecule suppresses amplification of the nucleic acid to which it is hybridized.

30. The method of claim 29, wherein said nucleic acid molecule or peptide nucleic acid molecule is in each case modified at the 5′-end thereof to preclude degradation by an enzyme having 5′-3′ exonuclease activity.

31. The method of claim 29, wherein said nucleic acid molecule or peptide nucleic acid molecule is in each case lack a 3′ hydroxyl group.

32. The method of claim 29, wherein the amplification enzyme is a polymerase lacking 5′-3′ exonuclease activity.

33. A method for detecting a cell proliferative disorder of a prostate peripheral zone, or for distinguishing between a transitional and a peripheral zone of origin of a prostate cell proliferative disorder, comprising:
a) obtaining, from a subject, a biological sample having subject genomic DNA;
b) extracting the genomic DNA;
c) contacting the genomic DNA, or a fragment thereof, comprising SEQ ID NO:1 or a sequence that hybridizes under stringent conditions to SEQ ID NO:1, with one or more methylation-sensitive restriction enzymes, wherein the genomic DNA is either digested thereby to produce digestion fragments, or is not digested thereby; and
d) determining, based on a presence or absence of, or on property of at least one such fragment, the methylation state of at least one CpG dinucleotide sequence of SEQ ID NO:1, or an average, or a value reflecting an average methylation state of a plurality of CpG dinucleotide sequences of SEQ ID NO:1, whereby at least one of detecting the prostate cell proliferative disorder, or distinguishing between a transitional and a peripheral zone of origin of the prostate cell proliferative disorder is, at least in part, afforded.

34. The method of claim 33, further comprising, prior to determining in step d), amplifying of the digested or undigested genomic DNA.

35. The method of claim 34, wherein amplifying comprises use of at least one method selected from the group consisting of: use of a heat resistant DNA polymerase as an amplification enzyme; use of a polymerase lacking 5′-3′ exonuclease activity; use of a polymerase chain reaction (PCR); generation of a amplificate nucleic acid carrying a detectable label; and combinations thereof.

36. The method of claim 35, wherein the detectable amplificate label is selected from the label group consisting of: fluorescent labels; radionuclides or radiolabels; amplificate mass labels detectable in a mass spectrometer; detachable amplificate fragment mass labels detectable in a mass spectrometer; amplificate, and detachable amplificate fragment mass labels having a single-positive or single-negative net charge detectable in a mass spectrometer; and combinations thereof.

37. The method of claim 36, comprising use of mass spectrometry for detecting amplificate, or detachable amplificate fragment mass labels.

38. The method of claim 37, wherein the mass spectrometry is selected from the group consisting of matrix assisted laser desorption/ionization mass spectrometry (MALDI), electron spray mass spectrometry (ESI), and combinations thereof.

39. The method of claim 33, wherein the biological sample obtained from the subject is selected from the group consisting of cell lines, histological slides, biopsies, paraffin-embedded tissue, bodily fluids, ejaculate, urine, blood, and combinations thereof.

40. A kit useful for detecting a cell proliferative disorder of a prostate peripheral zone, or for distinguishing between a transitional and a peripheral zone of origin of a prostate cell proliferative disorder, comprising:
a) a bisulfite reagent;
b) at least one nucleic acid molecule or peptide nucleic acid molecule comprising, in each case a contiguous sequence at least 9 nucleotides in length that is complementary to, or hybridizes under moderately stringent or stringent conditions to a sequence selected from the group consisting of SEQ ID NOS:1-5, and complements thereof; and
c) instructions, or directions for obtaining instructions for using the kit for detecting the prostate peripheral zone cell proliferative disorder, or for distinguishing between a transitional and a peripheral zone of origin of the prostate cell proliferative disorder.

41. The kit of claim 40, further comprising standard reagents for performing a methylation assay selected from the group consisting of MS-SNuPE, MSP, MethylLight™, HeavyMethyl™, COBRA, nucleic acid sequencing, and combinations thereof.

42. The method of any one of claims 1, 15 or 33, comprising use of the kit according to claim 36.
